(19) United States
(12) Patent Application Publication
(10) Pub. No.: US 2006/0142194 A1
(43) Pub. Date: Jun. 29, 2006

(54) Methods and compounds for treating brain amyloidosis

(75) Inventors: Roger Nitsch, Zollikon (CH); Johannes Pohlner, Hamburg (DE); Axel Wollmer, Zurich (CH); Hasan Mohajeri, Zurich (CH)

Correspondence Address: JACOBSON HOLMAN PLLC, 400 SEVENTH STREET N.W., SUITE 600, WASHINGTON, DC, 20004, US

(73) Assignees: Evotec NeuroSciences GmbH; Universitat Zurich

(21) Appl. No.: 11/274,493

(22) Filed: Nov. 16, 2005

Related U.S. Application Data
(63) Continuation of application No. 10/363,861, filed on Jul. 29, 2003, now abandoned.

(30) Foreign Application Priority Data
Sep. 19, 2000 (EA) 00120449.4

(51) Int. Cl. A61K 38/17

(52) U.S. Cl. 514/12

(57) ABSTRACT

The present invention discloses the use of (i) a fragment of the amyloid precursor protein (APP); or, (ii) a derivative of (i); or, (iii) a functional mimetic of (i) or (ii), in the preparation of a medicament for modulating CNS levels and/or activity of a member of the neprilysin family.

The present invention relates to the field of medicine, in particular to the field of neurodegenerative disease. The present invention provides for methods of eliciting clearance mechanisms for brain amyloid in patients suffering from neurodegenerative diseases, in particular Alzheimer's disease. Furthermore, this invention relates to the use of proteins and peptides effective in eliciting such mechanisms. Methods of screening for modulating agents of neurodegenerative disease are also disclosed.

Neurodegenerative diseases, in particular Alzheimer's disease, have a strongly debilitating impact on a patient's life. Furthermore, these diseases constitute an enormous health, social and economic burden. Alzheimer's disease is the most common age-related neurodegenerative condition affecting about 10% of the population over 65 years of age and up to 45% over age 85 (for a recent review see Vickers et al., Progress in Neurobiology 2000, 60:139-165). Presently, this amounts to an estimated 12 million cases in the US, Europe, and Japan. This situation will inevitably worsen with the demographic increase in the number of old people (“aging of the baby boomers”) in developed countries. The neuropathological hallmarks that occur in the brain of individuals suffering from Alzheimer's disease are senile plaques and profound cytoskeletal changes coinciding with the appearance of abnormal filamentous structures and the formation of neurofibrillary tangles. Both familial and sporadic cases share the deposition in brain of extracellular, fibrillary β-amyloid as a common pathological hallmark that is believed to be associated with impairment of neuronal functions and neuronal loss (Younkin S. G., Ann. Neurol. 37, 287-288, 1995; Selkoe, D. J., Nature 399, A23-A31, 1999; Borchelt D. R. et al., Neuron 17, 1005-1013, 1996). β-amyloid deposits are composed of several species of amyloid-β peptides (Aβ); especially Aβ₄₂ is deposited progressively in amyloid plaques. AD is a progressive disease that is associated with early deficits in memory formation and ultimately leads to the complete erosion of higher cognitive function. A characteristic feature of the pathogenesis of AD is the selective vulnerability of particular brain regions and subpopulations of nerve cells to the degenerative process. Specifically, the temporal lobe region and the hippocampus are affected early and more severely during the progression of the disease. On the other hand, neurons within the frontal cortex, occipital cortex, and the cerebellum remain largely intact and are protected from neurodegeneration (Terry et al., Annals of Neurology 1981, 10:184-192).

Genetic evidence suggests that increased amounts of Aβ₄₂ are produced in many, if not all, genetic conditions that cause familial AD (Borchelt D. R. et al., Neuron 17, 1005-1013, 1996; Duff K. et al., Nature 383, 710-713, 1996; Scheuner D. et al., Nat. Med. 2, 864-870, 1996; Citron M. et al., Neurobiol. Dis. 5, 107-116, 1998), pointing to the possibility that amyloid formation may be caused either by increased generation of Aβ₄₂, or decreased degradation, or both (Glabe, C., Nat. Med. 6, 133-134, 2000). Although these are rare examples of early-onset AD which have been attributed to genetic defects in the genes for APP, presenilin-1, and presenilin-2, the prevalent form of late-onset sporadic AD is of hitherto unknown etiologic origin. However, several risk factors have been identified that predispose an individual to develop AD, among them most prominently the epsilon4 allele of apolipoprotein E (ApoE) and the B-allele of cystatin C. The late onset and complex pathogenesis of neurodegenerative disorders pose a formidable challenge to the development of therapeutic agents.

Currently, there is no cure for AD, nor even a method to diagnose AD antemortem with high probability. However, β-amyloid has become a major target for the development of drugs designed to reduce its formation (Vassar, R. et al., Science 286, 735-41, 1999), or to activate mechanisms that accelerate its clearance from brain.

However, first experimental results by Schenk et al. (Nature, vol. 400, 173-177, 1999; Arch. Neurol., vol. 57, 934-936, 2000) suggest possible new treatment strategies for AD. The PDAPP transgenic mouse, which overexpresses mutant human APP (in which the amino acid at position 717 is phenylalanine instead of the normal valine), progressively develops many of the neuropathological hallmarks of AD in an age- and brain region-dependent manner. Transgenic animals were immunised with Aβ₄₂ either before the onset of AD-type neuropathologies (at 6 weeks of age) or at an older age (11 months), when amyloid-β deposition and several of the subsequent neuropathological changes were well established. Immunisation of the young animals essentially prevented the development of β-amyloid-plaque formation, neuritic dystrophy and astrogliosis. Treatment of the older animals also markedly reduced the extent and progression of these AD-like neuropathologies. It was shown that Aβ₄₂ immunisation results in the generation of anti-Aβ antibodies and that Aβ-immunoreactive monocytic/microglial cells appear in the region of remaining plaques. However, an active immunisation approach can entail serious side effects and hitherto unknown complications in human subjects.

Bard et al. (Nature Medicine, Vol. 6, Number 8, 916-919, 2000) report that peripheral administration of antibodies against amyloid β-peptide is sufficient to reduce amyloid burden. Despite their relatively modest serum levels, the passively administered antibodies were able to cross the blood-brain barrier and enter the central nervous system, decorate plaques and induce clearance of pre-existing amyloid. However, even a passive immunisation against amyloid b-peptide may cause undesirable side effects in human patients.

Iwata et al. (Nature Medicine, Vol. 6, number 2, 143-149, 2000) showed that the Aβ₁₋₄₂ peptide underwent full degradation through limited proteolysis conducted by neutral endopeptidase (NEP) similar or identical to neprilysin as biochemically analysed. Consistently, NEP inhibitor infusion resulted in both biochemical and pathological deposition of endogeneous Aβ₄₂ in brain. It was found that this NEP-catalysed proteolysis therefore limits the rate of Aβ₄₂ catabolism.

Neprilysin, also known as neutral endopeptidase-24.11 or NEP, is a 94 kD, type two membrane-bound Zn-metallopeptidase implicated in the inactivation of several biologically active peptides including enkephalins, tachykinins, bradykinin, endothelins and atrial natriuretic peptide. NEP is present in peptidergic neurons in the CNS, and its expression in brain is regulated in a cell-specific manner (Roques B. P. et al., Pharmacol. Rev. 45, 87-146, 1993; Lu B. et al., J. Exp. Med. 181, 2271-2275, 1995; Lu B. et al., Ann. N.Y. Acad. Sci. 780, 156-163, 1996). While type 2 NEP-transcripts are absent from the CNS, type 1 and type 3 transcripts are localized in neurons and in oligodendrocytes of the corpus callosum, respectively (Li C. et al., J. Biol. Chem. 270, 5723-5728, 1995). The Neprilysin family of proteases and endopeptidases comprises structurally or functionally homologous members of NEP such as the recently described NEP II gene and its isoforms (Ouimet T. et al., Biochem. Biophys. Res. Commun. 271:565-570, 2000), which are expressed in the CNS in a complementary pattern to NEP. A further member of this family is NL-1 (neprilysin like 1), a soluble protein efficiently inhibited by the NEP inhibitor phosphoramidon (Ghaddar G. et al., Biochem. J. 347: 419-429, 2000).

It is an object of the present invention to provide methods and materials which are suited, inter alia, for the development of a treatment for neurodegenerative diseases and for the identification of compounds useful for therapeutic intervention in such diseases. Based on the surprising finding that β-amyloid application elicits a neprilysin-mediated clearance mechanism for brain amyloid, the present invention sets out for providing such methods and materials as laid out in the claims section and described hereinafter.

The term “and/or” as used in the present specification and the claims implies that the phrases before and after this term are to be considered either as alternatives or in combination. For instance, the wording “determination of a level and/or an activity” means that either only a level, or only an activity, or both a level and an activity are determined.

The term “level” as used herein is meant to comprise a gage of, or a measure of the amount of, or a concentration of a transcription product, for instance an mRNA, or a translation product. The term “activity” as used herein can be understood as a measure for the ability of a transcription product or a translation product to produce a biological effect or a measure for a level of biologically active molecules. The terms “level” and/or “activity” as used herein further refer to either gene expression levels, gene activity, or enzyme activity.

In the present invention, a “fragment of the amyloid precursor protein” means a portion of the amyloid precursor protein (APP) which is less than the full amino acid sequence of APP and which has the property of increasing CNS levels and/or activity of a member of the neprilysin family, in particular neprilysin itself or its isoformes.

In the present invention, a “derivative of a fragment of the amyloid precursor protein” is a peptide or protein modified by varying the amino acid sequence of the fragment of the amyloid precursor protein, e.g. by manipulation of the nucleic acid encoding the fragment or by altering the fragment itself. Such derivatives of the natural amino acid sequence may involve insertion, addition, deletion or substitution of one or more amino acids, without fundamentally altering the property of the fragment of increasing CNS levels and/or activity of a member of the neprilysin family, in particular neprilysin itself or its isoforms.

In the present invention, a “functional mimetic” means a substance which may not contain a fragment of APP or a derivative thereof, and probably is not a peptide at all, but which has the property of increasing CNS levels and/or activity of a member of the neprilysin family, in particular neprilysin itself or its isoformes.

In one aspect, the present invention provides a pharmaceutical composition comprising an effective amount of monomers, multimers or aggregates of (i) fragments of the amyloid precursor protein (APP); or (ii) derivatives of (i) for treating or preventing a disease, in particular a neurodegenerative disorder. Preferably, the fragment is Aβ, in particular Aβ₁₋₄₀ or Aβ₁₋₄₂. The instant invention provides for the use of (i) a fragment of the amyloid precursor protein (APP); or, (ii) a derivative of (i); or, (iii) a functional mimetic of (i) or (ii), is suitable for the preparation of a medicament for modulating CNS levels and/or activity of a member of the neprilysin family, in particular neprilysin. To characterize mechanisms involved in Aβ clearance in vivo, according to the present invention, aggregated Aβ₄₂ was injected into brains of 11 week-old transgenic mice that expressed the disease-causing Swedish double mutation of APP (SwAPP) under the control of the murine PrP promoter, as well as into non-transgenic littermates (Hsiao K. K. et al., Neuron 15, 1203-1218, 1995; Hsiao K. et al., Science 274, 99-102, 1996). At this age no amyloid plaques in untreated transgenic SwAPP controls were observable applying standard amyloid staining methods. According to the present invention, it was unexpectedly found that Aβ₄₂ aggregates caused sustained increases in brain levels of NEP, and that these increases were associated with dramatically reduced brain concentrations of endogenous Aβ, as well as with prevention of brain amyloid plaque formation and with reduced astrogliosis. However, also mixtures of Aβ aggregates with monomers, or monomers themselves can be administered to modulate NEP activity or NEP levels. Increased brain levels of NEP may be due to an increased production or decreased degradation of NEP.

The above mentioned substances can be used for modulating neuronal or glial levels of a member of the neprilysin family. Preferably, the fragment or derivative of the amyloid precursor protein comprises a peptide substance of an amino acid sequence of the extracellular and the membrane-spanning region of APP. This fragment preferably comprises a peptide of 38 to 43 amino acid length obtainable by proteolytic activity of β- and/or γ-secretase. In particular, it comprises the Aβ₁₋₄₂ and/or the Aβ₁₋₄₀ peptide. A plurality of fragments in aggregated form, in particular in the form of fibrils, is preferably applied. The fragment of the amyloid precursor protein, or a derivative thereof, is preferably of synthetic nature. The aforementioned substances are particularly suited for increasing the CNS level and/or activity of neutral endopeptidase-24.11. Therefore, according to the present invention it is suggested to use at least one of these substances as a medicament for the treatment of neuro-degenerative disorders, in particular brain amyloidosis such as Alzheimer's disease (AD).

In a further aspect, a method for modulating CNS levels and/or activity of a member of the neprilysin family is provided comprising administering to a mammalian an effective amount of (i) a fragment of the amyloid precursor protein (APP); or (ii) a derivative of (i); or, (iii) a functional mimetic of (i) or (ii). One preferred means of administration is by intracerebral injection, but other means of administration, e.g. administration to the cerebrospinal fluid, or administration by oral or nasal means are also desirable.

In another aspect, a method for preventing and treating neurodegenerative disorders comprising administering to a mammalian an effective amount of (i) a fragment of the amyloid precursor protein (APP); or, (ii) a derivative of (i); or, (iii) a functional mimetic of (i) or (ii) to a region of the central nervous system (CNS) is provided. Said fragments or derivatives can be administered in their monomeric form, or in a multimer or aggregated form as explained above. The neurodegenerative disorder might be a brain amyloidosis, such as Alzheimer's disease. Said effective amount of (i) a fragment of the amyloid precursor protein (APP); or, (ii) a derivative of (i); or, (iii) a functional mimetic of (i) or (ii) can be administered by intracranial injection, or it can be administered to the cerebrospinal fluid (CSF). The administration might also be performed orally or nasally. As explained in detail above, the fragment or derivative of the amyloid precursor protein particularly comprises a peptide substance of an amino acid sequence of the extracellular and the membrane-spanning region of APP. Preferably, it comprises a peptide of 38 to 43 amino acid length obtainable by proteolytic activity of β- and/or γ-secretase. The fragment might comprise Aβ₁₋₄₂ peptide and/or Aβ₁₋₄₀ peptide. The fragment of amyloid precursor protein, or a derivative thereof, can be of synthetic nature. In particular, a plurality of fragments in aggregated form, in particular in the form of fibrills, is administered. Ideally compounds mimicking said above described activity are applied.

In still a further aspect, the invention provides a method for preventing and treating neurodegenerative disorders comprising administering to the central nervous system of a mammalian an effective amount of a nucleic acid coding for a member of the neprilysin enzyme family. In particular, the nucleic acid codes for a secretoric isoform of neprilysin. The method is suited for preventing and treating brain amyloidosis such as Alzheimer's disease. In this respect, the invention also provides a pharmaceutical composition comprising an effective amount of a nucleic acid coding for a member of the neprilysin enzyme family (e.g. a secretoric isoform of neprilysin) for treating or prevention a disease, in particular a neurodegenerative disorder.

The invention also provides different assay principles—biochemical and in particular cellular assays—for testing a compound, preferably screening a plurality of compounds, for modulating the production and secretion of a member of the neprilysin enzyme family. Due to practicability said assay principles should be homogeneous, preferably homogeneous high throughput assays. The substrates, inhibitors or target protease comprise at least one detectable label, preferably an optically detectable label which is luminescent, in particular fluorescent. The assay can then preferably be performed as a fluorescence energy transfer assay, a fluorescence lifetime assay, a fluorescence polarization assay, a fluorescence correlation spectroscopy assay, or a fluorescence intensity distribution assay (FIDA), or an assay based on other state of the art fluorescence techniques.

In a first aspect with regard to assay principles, the invention comprises the detection of an enzymatic activity of a member of the neprilysin familiy, or a level of the member of the neprilysin enzyme family, or its production, or its secretion, wherein a modulation of the enzymatic activity, or the level, or production, or secretion of the member of the neprilysin enzyme family in the presence of the compound in comparison to the enzymatic activity, or level, or production, or secretion in the absence of the compound indicates that the compound acts as a modulator. Using cellular assay systems, the amount of NEP produced can be determined e.g. by antibody binding using detectable, preferably fluorescently detectable antibodies. Alternatively, the binding of a detectable inhibitor to cell lines expressing NEP could provide a means to determine the level of NEP produced in response to a particular compound.

Preferably, the production of cell-membrane bound NEP or production and secretion of a NEP isoform can be determined via labelled antibody directed against NEP or its isoforms or by binding of detectable specific natural inhibitors such as spinorphin or synthetic inhibitors in the presence of library compounds to be screened. The invention therefore comprises cell lines, including but not limited to recombinant cell lines expressing NEP on the cell surface or secreting isoforms of NEP. The detection readout of these assays therefore comprises the NEP protein level, determined by binding assays using fluorescently labelled antibodies, bead-coupled antibodies or the determination of enzymatic activity using the below described procedures.

In a further assay principle, said assay comprises the steps of:
providing a substrate for a member of the neprilysin enzyme family;
incubating the substrate with (i) the member of the neprilysin enzyme family or a fragment thereof which fragment comprises the active center of the enzyme and (ii) a compound to be tested;
detecting an enzymatic activity and/or a level of the member of the neprilysin enzyme family, wherein a modulator of the enzymatic activity and/or the level of the member of the neprilysin enzyme family in the presence of the compound in comparison to the enzymatic activity and/or level in the absence of the compound indicates that the compound acts as a modulator.

This principle is suitable for both soluble biochemical assays and assays utilising cells expressing NEP family members or assays utilising soluble supports, such as beads, to which e.g. the substrate or the compound is bound.

In a further preferred embodiment, said substrate is bound to a soluble or suspendable support, preferably a bead. It might be advantageous that the member of the neprilysin enzyme family is integrated into a cell membrane, or a vesicular particle. However, it is also possible that the member of the neprilysin family is secreted by a cell, preferably a recombinant cell. Preferably, said member of the neprilysin family is neutral endopeptidase-24.11. Suitable substrates can be chosen from the group consisting of enkephalins, tachykinins, atrial natriuretic peptides, and synthetic peptides with a hydrophobic residue in the P1′ position. Possible substrates comprise intramolecularly quenched fluorogenic peptides structurally related, but not exclusively similar to enkephalin, substance P or similar peptidometric structures. Synthetic substrates such as the intramolecularly quenched fluorogenic peptide structurally related to leu-enkephalin, containing o-aminobenzoyl (Abz) and ethylendiamine 2,4-dinitrophenyl (EDDnp) groups at amino- and carboxyterminal amino acid residues have been described (Carvalho, M. et al. Analyt. Biochemistry 237, 167-173, 1995).

Another modification of this assay comprises neural substrates of the NEP protease or family members, e.g. substance P, encephalins etc. which were fluorescently labelled and covalently linked to microbeads. The fluorescence intensity of these beads in solution can be assessed using fluorescent techniques, e.g. FIDA or polarization or other state of the art detection techniques. In absence of protease activity the beads display the maximum fluorescent intensity. Beads partially stripped of the fluorescently labelled substrate due to enzymatic cleavage display a lower fluorescence intensity. This decrease in fluorescence intensity directly correlates with NEP protease activity. The procedure can be performed as follows. Firstly, equal amounts of protease will be dispensed in test wells together with compound libraries. Secondly, the beads will be added and FIDA analysis performed. A modulation of the protease activity will result in an altered fluorescent signal per bead using FIDA technology.

It is also possible, using this type of assay, to determine inhibition or activation of endopeptidase cleavage of fluorescently labelled oligopeptides. The endopeptidase is placed in solution with library compound and suspendable solid supports that are coated with fluorescently labelled oligopeptide substrate. Upon cleavage of the substrate, fluorescently labelled product is released from the supports. The amount of fluorescence that remains bound to the bead increases if an inhibitor is present.

A further assay principle comprises the use of FRET (Fluorescent resonance energy transfer) phenomenon. FRET can be observed e.g. with substrates which have been labelled N-terminally with Cy5 or substitutes and C-terminally with RhGreen or substitutes. The exitation of RhGreen at 488 nm results in an energy transfer towards Cy5, since both dyes are in close proximity. Upon addition of NEP the peptide is cleaved and the energy transfer interrupted. After cleavage the fluorescent intensity decreases due to a decreasing Cy5 emission.

In a further embodiment, the assay comprises the addition of a known inhibitor of the member of the neprilysin family before detecting said enzymatic activity. Suitable inhibitors are e.g. phosphoramidon, thiorphan, spinorphin, or a functional derivative of the foregoing substances. The compound identified as an activator is for the treatment of neurodegenerative disorders, in particular brain amyloidosis such as Alzheimer's disease (AD).

In a further embodiment of a substrate-based assay, the invention relates to competition assays screening for compounds which disrupt the interaction of the brain specific endogenous inhibitor spinorphin or other natural and synthetic inhibitors and the NEP target protease family. Brain specific endogenous inhibitors such as Spinorphin can be used to search for compounds enhancing protease activity and screen for compounds relevant in the CNS. The usage of inhibitors, however, shall not be limited to naturally occurring inhibitors. The principle of this assay is a competition between the inhibitor and the substrate for the protease NEP and/or isoenzymes. The protease activity is determined by the conversion of a fluorescently labelled substrate as described above, utilizing an unlabelled inhibitor in the assay. A labelled substrate for the protease such as substance P is incubated with the inhibitor and NEP. This equilibrium will result in a baseline fluorescent signal intensity. The readout is an alteration of fluorescent signal in the presence of compound interfering with the protease/inhibitor complex. This assay also includes variations of this principle using substrates bound to beads, soluble substrates and/or fixed substrates, being fluorescently labelled or detected via labelled antibodies, and similar detection procedures. Summarized, this assay type screens for compounds able to interfere with NEP-inhibitor-complex, resulting in displacement of inhibitor and thereby enabling binding and cleavage of the substrate.

In a general sense, assays according to the invention measure the production and secretion of NEP. The compound might e.g. act on a receptor, such as a cell membrane receptor, or influence a signalling pathway within a cell ultimately resulting in stimulating the production and secretion of a member of the NEP family. The compound may influence the transcriptional level, post-transcriptional modifications, increased transcript stability and increased protein stability e.g. via inhibition of degradation as well as inhibitors, or modulators of inhibitors of said NEP protease.

Therefore, in another aspect, the invention provides an assay for testing a compound, preferably of screening a plurality of compounds, useful for transcriptional activation of a member of the neprilysin family, comprising:
providing a recombinant cell containing a reporter gene construct wherein the reporter gene construct contains (i) a transcriptional control element, preferably a CNS specific control module known to regulate the transcription of a gene coding for the member of the neprilysin family and (ii) a reporter gene that encodes a reporter gene product and is in operative association with the transcriptional control element;
incubating the recombinant cell with a compound to be tested;
comparing the amount of reporter gene product expressed in a recombinant cell in the presence of the compound with the amount of reporter gene product in the absence of the compound, wherein an increase in the amount of reporter gene product in the presence of the compound in comparison to the amount of reporter gene product in the absence of the compound indicates that the compound is a transcription activator.

Preferably, said member of the neprilysin family is neutral endopeptidase-24.11 and said transcriptional control element comprises the regulatory cis-elements of a gene coding for endopeptidase-24.11.

Said assay should be homogeneous, preferably a homogeneous high throughput assay. The reporter gene product should be luminescent, in particular fluorescent, or produce another optically detectable read-out. However, any other type of detectable label can also be used. The reporter gene product can e.g. be an enzyme which interacts with a substrate to produce an optically detectable read-out. The compound identified as a transcription activator may act on different levels of the cellular signaling cascade and is suited for the treatment of neurodegenerative disorders, in particular brain amyloidosis such as Alzheimer's disease (AD).

A further aspect of this invention relates to screening assays including high throughput screening of chemical compounds modulating the production and secretion of endogenous inhibitors of NEP, such as spinorphin. A compound might e.g. act on a receptor, such as a cell membrane receptor, or influence different levels of a signaling cascade ultimately resulting in an altered, preferably decreased production and/or secretion of endogenous brain specific inhibitors of NEP family members. Different assay principles can be applied for testing a compound, preferably screening a plurality of compounds, for production and secretion of endogenous inhibitors of the neprilysin enzyme family, such as the CNS specific inhibitor spinorphin using biochemical or preferably cellular assays.

The aim of these assays is the reduction of the production or secretion of endogenous inhibitors of the neprilysin enzyme family. The readout of the assay comprises e.g. an increase of the enzymatic activity and/or level of neprilysin or family members in the presence of the compound in comparison to the enzymatic activity and/or level in the absence of the compound, indicating that the compound acts as an inhibitor of an endogenous inhibitor such as spinorphin. Using cellular assay systems, alternatively the amount of sphinorphin produced can be determined by antibody binding assays using labelled, preferably fluorescently labelled antibodies. Alternatively, the binding of the labelled target protease NEP or labelled NEP peptide fragments containing the spinorphin binding pocket can be used. Incubation of these labelled baits with cell lines could provide a means to determine the level of spinorphin produced in response to a particular compound.

In another aspect, the invention provides an assay for testing a compound, preferably of screening a plurality of compounds, for decreasing production and secretion of endogenous inhibitors of NEP family members, e.g. Sphinorphin. The assay comprises the following steps: (i) incubating cells expressing and secreting the endogenous inhibitor with a compound to be tested, and (ii) determining the production and/secretion of endogenous inhibitors using e.g. antibody labeling techniques. However, it is known to the person skilled in the art that secreted inhibitor can also be detected e.g. through binding of NEP target protease.

In another aspect, the present invention provides a method for producing a medicament comprising the steps of (i) identifying a compound as either a modulator of a member of the neprilysin family, or as a transcriptional activator of a member of the neprilysin family, or as modulator of production, preferably secretion of a member of the neprilysin family, or as a modulator of production, preferably secretion of inhibitors of a member of the neprilysin family according to any of the herein described methods and assays and (ii) admixing the compound with an appropriate pharmaceutical carrier.

In a further embodiment, the present invention provides for a medicament obtainable by any of the herein described methods and assays.

In another embodiment, the instant invention provides for a medicament obtained by any of the herein described methods and assays.

Other features and advantages of the invention will be apparent from the following description of figures and examples.

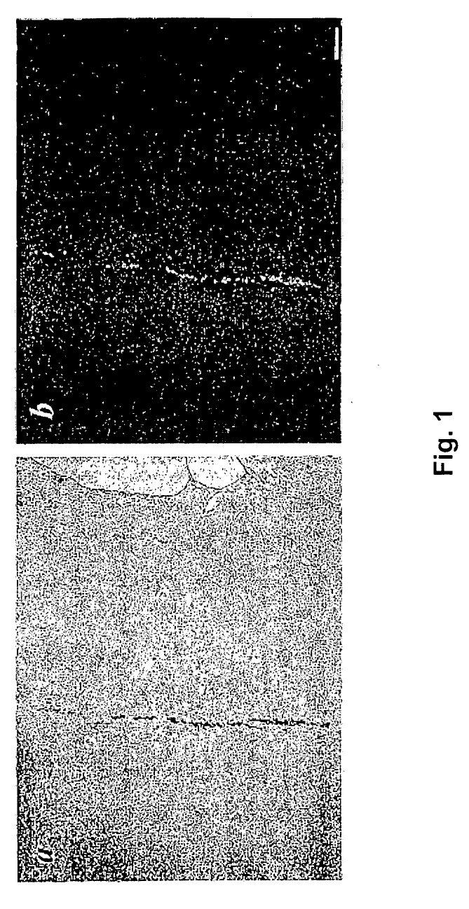

FIG. 1 shows the needle tract of injected Aβ₄₂ aggregates. Synthetic Aβ₄₂ was aggregated for 2 days in PBS at 37° C. before injections into the brain cortex. Eleven weeks after the Aβ₄₂ injection, the needle tract was still immunoreactive for the monoclonal antibody 4G8 (a). Injected material was also thioflavin S-positive indicating fibrillar structure (b). Scale: 200 μm.

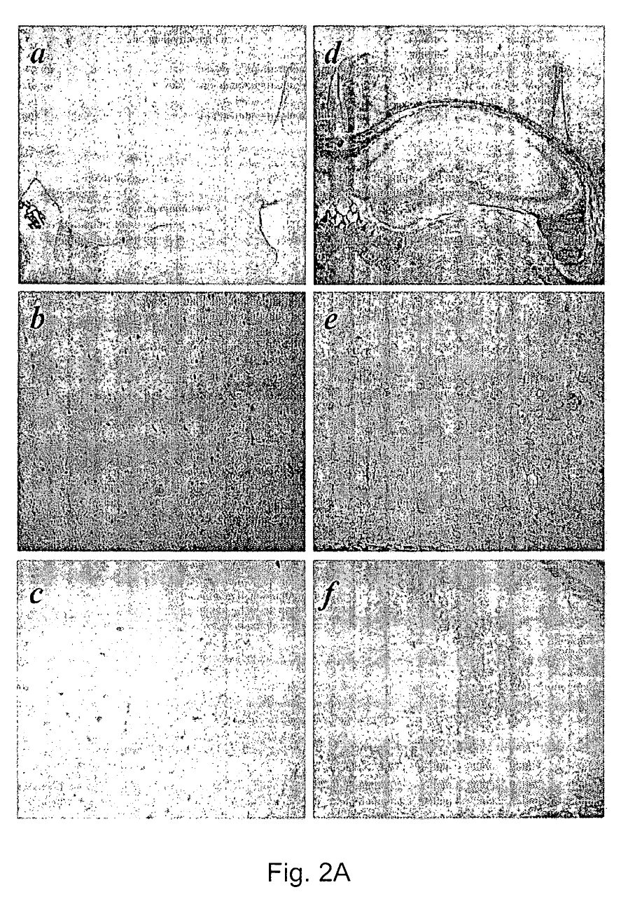

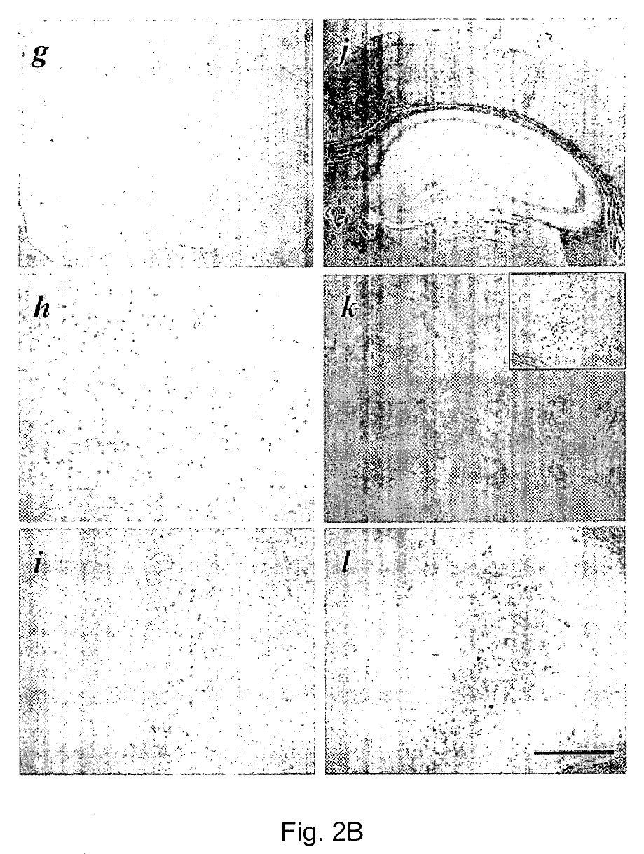

FIG. 2 shows the Aβ₄₂ increased neuronal NEP-immuno-reactivity in wild-type (a-f) and SwAPP mice (g-1). Aβ₄₂-induced increases in NEP-immunoreactivity were detected in cortex, hippocampus, and axonal tracts of Aβ₄₂ injected wild-type (d-f) and SwAPP (j-i) mice. In contrast, PBS injected wild-type mice (a-c) or SwAPP mice (g-i) showed only weak and diffuse NEP-staining in these structures. NEP staining was most intense around and near the injection site (j, inset in k). Scale: 1 mm for a,d,g,j; 200 μm for b,c,e,f,h,i,k,l; and 40 μm for inset in k. 20 weeks after injections.

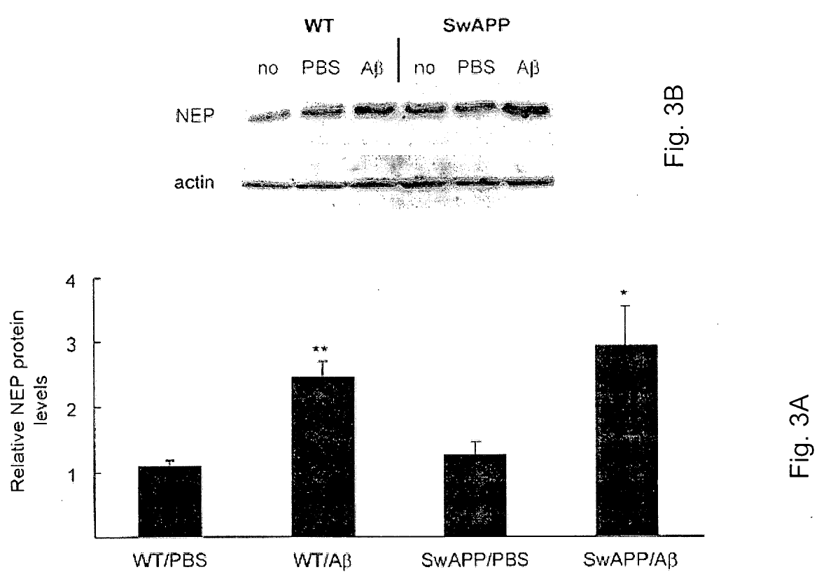

FIG. 3 illustrates that levels of NEP increased as early as one week after Aβ₄₂ injection in brain extracts of transgenic SwAPP mice and wild-type littermates. Actin was used to control for equal loading (a). Densitometric analyses of the Western blot (b) revealed a significant increase of NEP protein levels after Aβ₄₂ injections. The intensity of each NEP and actin bands was measured using the NIH-Image software. The NEP/actin-ratios were assessed for each mouse and normalised to the wild-type values set to 100%. Data represent means of each group+SEM * p≦0.05, ** p≦0.01, Mann-Whitney U test. n=3 for each group.

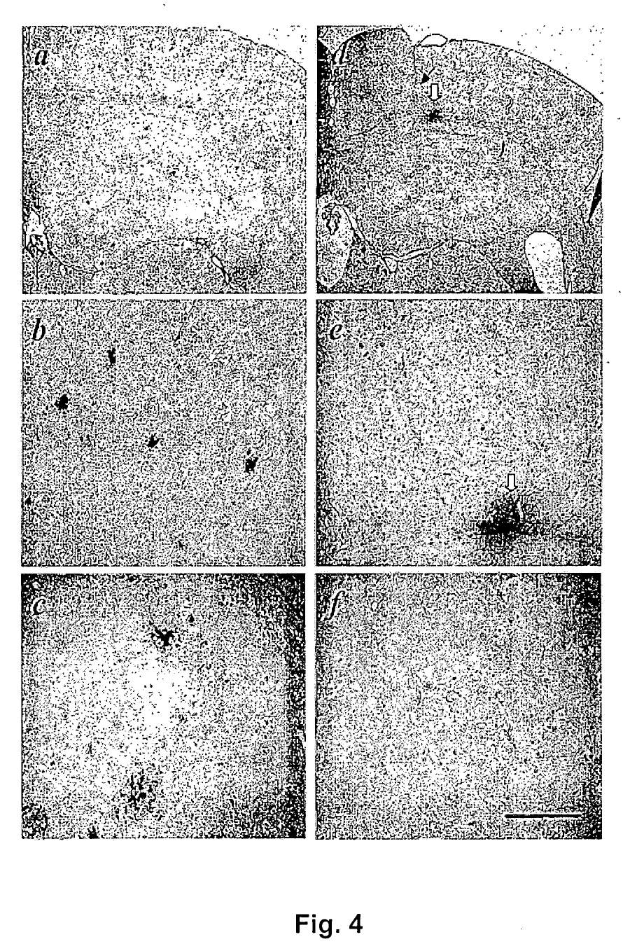

FIG. 4 depicts the Aβ₄₂-mediated inhibition of amyloid plaque formation in transgenic SwAPP mice. Twenty weeks after injection of Aβ₄₂ aggregates into cortex of SwAPP mice (d-f), a significant reduction of 4G8-positive amyloid plaques was observed throughout the brain (d), in cortex (e) and hippocampus (f). Whereas age-matched untreated SwAPP mice (a-c) exhibited numerous 4G8 positive plaques in cortex (a-d), only careful examination of treated SwAPP mice revealed the presence of some amyloid plaques. Scale: 1 mm for a, d; 200 μm for b, c, e, f.

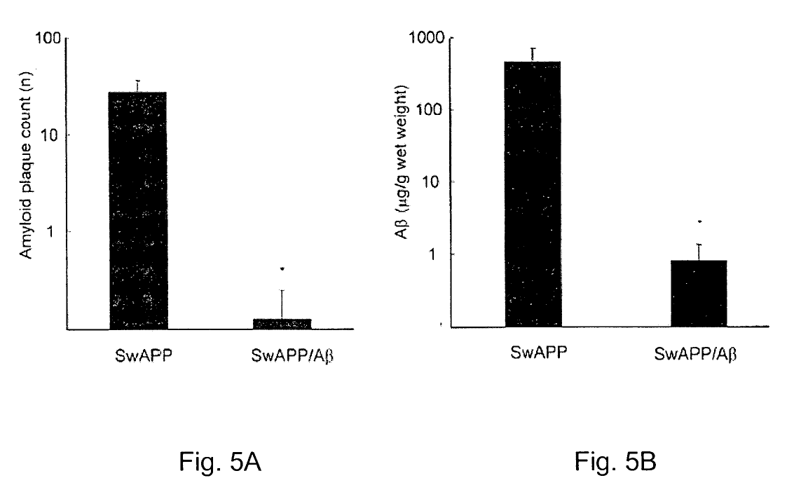

FIG. 5 shows that numbers of amyloid plaques and brain levels of Aβ₄₂ were significantly lower 20 weeks after intracranial injection of Aβ₄₂. Randomly chosen sections of Aβ₄₂ injected SwAPP mice and untreated age-matched SwAPP littermates were examined for the presence of 4G8-positive amyloid plaques in randomly selected visual fields covering cortical areas equal to 5 mm² on 5 μm thick paraffin sections (a). Plaque counts were significantly reduced in SwAPP mice after Aβ₄₂ treatment (*: p≦0.05, Mann-Whitney U test). Aβ ELISA performed on formic acid extracted total Aβ (b) revealed that 20 weeks following the injections, Aβ levels in Aβ₄₂-injected SwAPP mice were also significantly reduced (*: p≦0.05, Mann-Whitney U test) as compared to untreated SwAPP littermates (b). Mean values+SD.

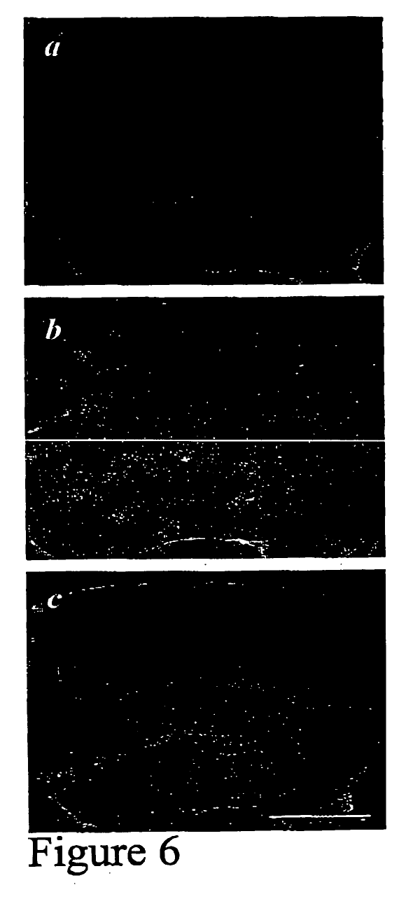

FIG. 6 illustrates that Aβ₄₂ reduced astrogliosis associated with amyloid formation in SwAPP mice. Representative sections through the cortex and hippocampus are shown. 20 weeks after Aβ₄₂ injections, the overall astrogliosis was lower in SwAPP mice treated with Aβ₄₂ (c), as compared to untreated SwAPP littermates (b), but higher than wild-type littermates (a). Please note the nearly complete absence of astrogliosis in cortex of treated SwAPP mice (c) whereas many reactive astrocytes could be found in hippocampus. Scale: 1 mm.

### EXPERIMENTS

Animals: Transgenic mice expressing the AD-causing Swedish double mutant of the human APP gene were generated and bred as described previously (Hsiao K. K. et al., Neuron 15, 1203-1218, 1995; Hsiao K. et al., Science 274, 99-102, 1996). All mice used in this study were the progeny of a single SwAPP male, crossbred to wild-type littermates. The colony was housed under a light cycle of 12 hours with dry food and water ad libitum. The presence of the transgene was determined by PCR on genomic DNA isolated from tail biopsies using specific primers GTG GAT AAC CCC TCC CCC AGC CTA GAC CA and CTG ACC ACT CGA CCA GGT TCT GGG T to the transgene. At 11 weeks of age, mice were anesthetised, 1 μl fibrilar Aβ₄₂ or PBS was stereotaxically injected unilaterally into the parietal cortex above the hippocampus (350 μM stock concentration), within 1 minute. After the injection, the needle was left in place for 1 more minute, and then was slowly withdrawn over a period of 1 minute. Fibrillar aggregated synthetic Aβ₄₂ was prepared by resuspending lyophilized Aβ₄₂ (Bachem) in PBS, pH 7.4 by shaking for 48 hours at 37° C.

Preparation of tissue: For combined Western blot analyses and immunohistochemistry, mice were deeply anesthetized and were perfused transcardially using ice-cold PBS (pH 7.4). Frontal cortices of both hemispheres were homogenized in lysis buffer containing 1% Triton X-100, 10 mM Tris, 250 mM sucrose, and 1× complete proteinase inhibitor cocktail, pH=8 (Roche, Switzerland). The remaining brain tissue was fixed over night in 4% paraformaldehyde solution at 4° C. and washed several times in PBS and embedded in paraffin for immuno-histochemistry. For combined immunocytochemistry and Aβ-ELISA, mice were perfused with 4% paraformaldehyde and postfixed in the same solution over night. The caudal parts of the brains reaching from intraaural 1 to intraaural −2 (Franklin, K. B. J, Paxinos, G. Academic Press, 1997) were dissected and prepared for Aβ ELISA. The remaining brain tissue was embedded in paraffin and then processed for immunohistochemistry. Five μm thick paraffin sections were probed with antibodies against NEP, IDE (Insulin degrading enzyme), ACE (Angiotensin converting enzyme), endopeptidase 24.15 protein, and GFAP (glial fibrillary protein) according to providers' protocols and counterstained with nuclear fast red (Fluka, Switzerland).

Western-blot analysis: Protein concentration of brain extracts were determined with the Bredford protein assay and equal amounts of protein (60 μg) were loaded to each lane of 8% acrylamid gels in Laemmli buffer. After electrophoresis proteins were blotted on nitrocellulose membranes (Amersham). Membranes were probed for NEP using the monoclonal antibody 56C6 at a dilution of 1:50. Blots were stripped in a buffer containing 2% SDS, 62.5 mM Tris (pH 6.8) and 100 mM β-mercaptoethanol at 50° C. for 30 minutes. After stripping the membrane were probed sequentially with anti-Alzheimer precursor protein A4 antibody (clone 22C11, 1:1000), IDE-1 (1:800), ACE (1:200), and the polyclonal antibody to 24.15 protein (1:600). Finally, staining for actin (1:1000) was performed as a loading control. Antibodies to NEP (clone 56C6), ACE, actin, and GFAP were purchased from Novacastra, QED Bioscience, Sigma, and InnoGenex, respectively. Anti Alzheimer precursor protein A4 antibody (clone 22C11) was purchased from Boehringer. IDE-1 antibody was a gift from Drs. Vekrellis and Selkoe (Vekrellis K. et al., J. Neurosci. 20, 1657-1665, 2000), and antibodies to 24.15 were gifts from Dr. C. Abraham (Yamin, R. et al., J. Biol. Chem. 274, 18777-18784, 1999).

Aβ-ELISA: For quantitation of Aβ content in mouse brain, protein extracts of fixed brain tissue reaching from the stereotaxic coordinates intraaural 1 to intraaural −2 were prepared. Tissue samples were homogenized in a 25-fold wet weight amount of 70% formic acid by repeated passage through a 23G injection needle. Homogenates were centrifuged at 200000 g for 1 hour at 4° C., and supernatants were neutralized by adding the 20-fold volume 1 M Tris base. Microtiter plates (Maxi sorb, Nunc) were coated with 150 μl of monoclonal mouse antibody 22C4, directed against the C-terminus of Aβ₄₀ and Aβ₄₂, at a concentration of 20 μg/ml PBS. Plates were blocked with 1% BSA, 1% gelatin in 100 mM Tris, 5 mM EDTA and 0.1% Tween 20 pH 7.6 for 4 hours at 37° C., washed three times with PBS containing 0.02% Tween 20. 150 μl diluted samples or standards (Aβ₄₀ Bachem) were incubated overnight, washed, and detected with biotinylated monoclonal mouse antibody 6H1 directed against amino acids 1-17 of human Aβ (Evotec Neurosciences GmbH) incubated at a concentration of 1 μg/ml in blocking buffer for 2 hours at 37° C., and visualised with a peroxidase reaction using tetramethylbenzidine as substrate, and detection at 450 nm. The linear range (R²=0.98) of the used ELISA system was between 0-100 ng Aβ/ml. Samples were adjusted to linear range by serial dilution.

Aβ₄₂ increased brain concentrations of NEP: To determine whether Aβ₄₂ is involved in the regulation of NEP levels in vivo, Aβ₄₂ was injected into the brains of SwAPP mice and littermate controls, and NEP was analyzed by immunohistochemistry. A 350 μM solution of Aβ₄₂ was aggregated under shaking conditions and one single intracranial injection of 1 μl of the resulting suspension generated thioflavin S-positive and 4G8-immunoreactive deposits that were detectable for at least 11 to 20 weeks following the injections (FIG. 1, a, b, FIG. 4 d, e, open arrow). Aβ₄₂ increased NEP in neurons throughout the brains of both SwAPP and wild-type littermates (FIG. 2 d-f, j-l), with strongest NEP immunoreactivity in cortical neurons surrounding the injection site (FIG. 2j, inset in k). Strongly stained NEP-immunopositive neurons were present both 11 and 20 weeks following one single Aβ₄₂ injection (FIG. 2 d-f, j-l). Twenty weeks after Aβ₄₂ injections, NEP immunoreactivity was high in somatodendritic compartments of many pyramidal cells of the cerebral cortex of all cortical layers (FIG. 2 d, e, j, k), in the cell bodies of pyramidal neuronal within the CA1 and CA3 regions of the hippocampus (FIG. 2 f, l), as well as in axonal tracts of the corpus callosum (FIG. 2 d, j) as well as in a few glial cells in the hippocampus (FIG. 2 d, j). In contrast, in SwAPP mice as well as in wild-type littermates injected with PBS vehicle alone, neuronal NEP staining was weak and diffuse (FIG. 2 a-c, g-i). Together these data show that Aβ₄₂ increased neuronal levels of NEP in most brain regions. To confirm this finding, we next analysed brain proteins prepared from Aβ₄₂-injected mice extracts by Western blotting. While endogenous levels of NEP were similar in uninjected or PBS-injected SwAPP mice or wild-type littermates, these were significantly higher in Aβ₄₂-injected mice as early as one week after Aβ₄₂ injection (FIG. 3a). Densitometric analyses of several blots suggested that Aβ₄₂ increased brain concentrations of NEP 2- to 4-fold (FIG. 3b).

Aβ₄₂-induced increase in NEP was associated with reduced amyloid plaque formation: To determine whether the Aβ₄₂-induced increase in neuronal NEP was associated with reduced amyloid plaque formation in transgenic SwAPP mice, the number of brain amyloid plaques stained either by thioflavin S or by immunohisto-chemistry at 1, 11, and 20 weeks after one single injection of Aβ₄₂ was counted. As expected, all untreated SwAPP mice (6/6) tested at end of the experiment (31 weeks of age) exhibited numerous thioflavin S positive amyloid plaques that also immunoreacted with the monoclonal antibody 4G8 (Serotec) directed against the amino acids 17-24 of human Aβ. In particular, amyloid plaques were abundant throughout the cerebral cortex (FIG. 4 a, b) and, to a somewhat lesser extend, in the hippocampus (FIG. 4c). Other Aβ specific antibodies including 6E10 (Serotec), 6H1 (Evotec Neurosciences GmbH), and 9G10 (Evotec Neurosciences GmbH) gave identical results. In striking contrast, SwAPP mice that had received single injections of aggregated Aβ₄₂ were completely free of thioflavin S- or 4G8-positive amyloid plaques at 31 weeks of age, or 20 weeks after the injection (4/4) (FIG. 4 d-f). In these mice, 4G8 immunoreactive material was observed only around the needle tract; this material represented the previous injected aggregated synthetic peptide (FIG. 4 d, e, open arrow).

To be sure that no amyloid plaques were missed in the mice, every 50^{th} coronal section (250 μm apart) of all mice was stained, beginning with a random section of the olfactory bulbs. Only a few faintly 4G8-immunoreactive structures in SwAPP mice were found at 20 weeks after Aβ₄₂ injections, in addition to occasional but remarkably few, vascular amyloid deposits (not shown). Existing plaques in 3 representative sections of each mouse were counted. Plaque counts showed a marked significant decrease of 4G8 stained amyloid plaques in Aβ₄₂-treated SwAPP mice as compared to untreated littermates (FIG. 5a). To determine whether the reduced amyloid pathology was associated with reduced concentrations of transgenic Aβ, human Aβ was measured by a sandwich ELISA that specifically recognized intact human Aβ (Nitsch, R. M. et al., Ann. Neurol. in Press). As expected, no human Aβ was detected by ELISA in wild-type mice. Aβ levels measured in untreated control SwAPP mice (n=4) were significantly higher than in Aβ₄₂-treated SwAPP mice (n=4) (FIG. 5b). Because this sandwich ELISA detected only intact Aβ, but not cleavage products, these data are compatible with the possibility that intracranial injection of exogenous Aβ₄₂ fibrils accelerated the proteolytic degradation of endogenous, transgenic human Aβ. To exclude that APP levels changed in response to Aβ₄₂ injections, Western blots of protein extracts were performed—no difference among Aβ₄₂- or vehicle treated, or untreated transgenic mice was found (not shown).

Prevention of amyloid formation was accompanied by reduced reactive astrocytosis: Because brain amyloidosis in SwAPP mice is accompanied by reactive astrocytosis, GFAP-reactive astrocytes were analysed in response to the Aβ₄₂-injection protocol (FIG. 6 a-c). Twenty weeks after injection, GFAP staining of reactive astrocytes was significantly lower in cerebral cortices of Aβ₄₂-injected SwAPP mice (FIG. 6c) as compared to untreated SwAPP littermates (FIG. 6b) but significantly higher than in wild-type littermates (FIG. 6a). Moreover, amyloid plaque-associated clusters of GFAP-positive cells commonly found in SwAPP mice were completely absent in response to Aβ₄₂ injections, paralleling the absence of amyloid plaques in Aβ₄₂-treated mice (FIG. 4 d-f). GFAP-positive astrocytes were, however, abundant in hippocampi of Aβ₄₂-treated SwAPP mice, despite the significant reduction of amyloid plaques (FIG. 6c). Similar activation of astrocytes in brain regions unaffected by amyloid plaque formation is a well established finding in transgenic mice that overexpress disease-causing APP mutants (Schenk D. et al., Nature 400, 173-177, 1999). It may be explained by high sensitivity of hippocampal astrocytes to unknown toxic activities related to the expression of the SwAPP transgene; these may well be independent of amyloid plaque formation.

Prevention of amyloid formation was independent of IgG: To determine whether an IgG-mediated immune response similar to that observed by Schenk et al. (Nature 400, 173-177, 1999) was involved in the prevention of amyloid formation described here, the mice were tested for the presence of endogenous immunoglobin antibodies at the amyloid injection sites, and on the few plaques that had developed after Aβ₄₂ injections. By using immunochemistry with mouse-specific anti-immunoglobulin antibodies, no staining of the injected amyloid remaining around the needle tract was found, or at the few 4G8 immunoreactive structure that occasionally was observed in our Aβ₄₂-injected mice. This was in sharp contrast to peripherally immunised mice that had abundant IgG-positive plaques (not shown). These data strongly suggest that the mechanisms involved in prevention of amyloid formation differ between the single intracerebral injections of Aβ₄₂ fibrils and the peripheral immunisation protocols with added adjuvants and repeated boosts.

Role of other Aβ degrading enzymes: To determine whether other Aβ-degrading proteases were involved in preventing amyloid formation in the above described mice, brain tissue levels of insulin-degrading enzyme (IDE), a 100 kD Zn-metallo-proteinase that is a major Aβ-degrading enzyme in tissue culture (Qiu W. Q. et al., J. Biol. Chem. 273, 32730-32738, 1998; Vekrellis K. et al., J. Neurosci., 20, 1657-1665, 2000) were analysed by immunohistochemistry and Western blotting. We used the identical brain protein extracts and sections adjacent to those used for NEP immunohistochemistry for staining with the IDE-1 antibody (Vekrellis K. et al., J. Neuroci., 20, 1657-1665, 2000), and observed no differences in tissue levels of IDE in response to Aβ₄₂ injections with either technique (not shown). Moreover, metalloprotease 24.15, a recently identified as a Aβ-degrading enzyme (Yamin R. et al., J. Biol. Chem. 274, 18777-18784, 1999), was also unchanged in response to Aβ injections. Together, these data do not exclude possible roles of IDE and 24.15 in degrading Aβ in vivo, but they clearly suggest an important role of NEP in preventing amyloid formation in our experimental model. As an additional control experiment, we tested brain tissue levels of angiotensin converting enzyme (ACE), an unrelated neuronal Zn-metalloendo peptidase (Barnes N. M. et al., Eur. J. Pharmacol. 200, 289-292, 1991; Alvarez R. et al., J. Neurol. Neurosurg. Psychiatry 67, 733-736, 1999; Amouyel P. et al., Ann. N.Y. Acad. Sci. 903, 437-441, 2000) with no known affinity to Aβ (McDermott J. R. and Gibson A. M., Neurochem. Res. 22, 49-56, 1997). ACE levels, too, did not differ between treated and untreated mice at any examined time point following an Aβ₄₂ injection.

The amyloid cascade hypothesis is a predominant theory for the pathophysiology of AD (Selkoe D. I., Nature 399, A23-A31, 1999). It claims that the overproduction of Aβ or the failure to remove it is crucial to the development of AD, because both possibilities can lead to the formation of amyloid plaques and to neuronal damage. The amyloid theory gained strength by the observation that mutations in APP, PS1, and PS2 genes cause alterations in APP processing, and accelerate production of the highly aggregating Aβ₄₂. Moreover the possible direct role of PS1 in γ-secretase processing and in Aβ production support this hypothesis. Aβ₄₂ fibrils are likely formed by initial “seeding”, followed by precipitation of further Aβ molecules into amyloid plaques (Jarrett I. T. et al., Cell 73, 1055-1058, 1993). The data according to the present invention clearly show that injection of synthetic fibrillar Aβ₄₂ did not accelerate amyloid formation in SwAPP mice. In contrast, this treatment activated a mechanism that was associated with increased expression—or decreased turnover—of NEP, a metalloproteinase that can degrade Aβ₄₂ in mammalian brain (Iwata N. et al., Nat. Med. 6, 143-150, 2000). This activation, in turn, was associated with a dramatic reduction in brain tissue levels of Aβ, and almost complete inhibition of amyloid plaque formation. These data are possibly at variance with those reported by Kane et al. (J. Neurosci. 20, 3606-3611, 2000) who found evidence for accelerated amyloid plaque formation after intracerebral infusion of AD brain extracts into SwAPP mice. This apparent discrepancy is most likely due to the fact that Kane et al. injected a complex mixture of human brain extracts into SwAPP mice whereas our injected material consisted of pure synthetic Aβ₄₂ fibrils and vehicle only. This interpretation is further supported by the finding that the “seeding effect” caused by brain extracts occurred also after immunodepletion of the extracts from Aβ, (Larry Walker, Alzheimer meeting, Washington D.C. 2000—Neurobiology of Aging; abstract) strongly arguing that the effects of brain extracts on brain amyloid formation are principally different than these described here. Because inhibition of NEP activity in vivo can induce brain amyloid plaque formation, it is possible that endogenous NEP inhibitors including the heptapeptide spinorphin, can slow Aβ degradation in vivo (Iwata N. et al., Nat. Med. 6, 143-150, 2000; Jarrett J. T. et al., Cell 73, 1055-1058, 1993), and thus accelerate amyloid formation. No data exist of the levels of spinorphin in AD, it is therefore impossible to estimate whether spinorphin contributed to the “seeding” of amyloid observed by Kane M. D. et al. (J. Neurosci. 20, 3606-3611, 2000).

Aβ can be cleared from ventricular CSF into blood within minutes after intraventricular injection (Ghersi-Egea J. F. et al., J. Neurochem. 67, 880-883, 1996), possibly via receptor-mediated transport through ventricle and choroid plexus cells (Zlokovic B. V., Proc. Natl. Acad. Sci. USA 93, 4229-4234, 1996; Mackic J. B. et al., J. Clin. Invest. 102, 734-743, 1998). These data are consistent with two observations we made during this study, a sustained astrogliois in hippocampus along with higher 4G8 and NEP immunoreactivity in hippocampus and fimbria of Aβ₄₂-treated SwAPP mice. Taken together, these data suggest an active role of the hippocampus and the ventricular system in clearing Aβ from the brain.

The mechanism that couples Aβ₄₂ to increased NEP levels is unclear, possibilities include a soluble signaling factor that can reach neurons throughout the brain, either directly by a paracrine mechanism or via the circulation. Because altered gene expression in response to unilateral neuronal injury is often unrestricted to the ipsilateral site and is also found contralateraly (Henken D. B. et al., Neuro-science 39, 733-742, 1990; Eckert A. et al., Pain 83, 487-497, 1999), increased NEP gene expression may be involved in causing the elevated brain levels of NEP. The synthetic Aβ aggregates injected into mouse brains were remarkably stable over 11 to 20 weeks following the injection and seems to be resistant to the clearing mechanism triggered here. This may—at least in part—explain the long-term increases in NEP levels in response to Aβ injections. Our data suggest the possibility that the increase in NEP is related to inhibition of amyloid plaque formation by decreasing brain levels of Aβ. As a consequence, amyloid plaque-related astrogliosis was also reduced.

1-38. (canceled)

39. A method of treating a neurodegenerative disorder, wherein the neurodegenerative disorder is brain amyloidosis or Alzheimer's disease, comprising increasing neutral endopeptidase-24.11 activity in a patient by administering to the patient a medicament containing
i) a fragment of the amyloid precursor protein,
ii) a derivative of the amyloid precursor protein obtained by varying the amino acid sequence of the fragment, or
iii) a functional mimetic of the fragment or the derivative.

40. The method of claim 39 wherein the neurodegenerative disorder is brain amyloidosis.

41. The method of claim 39 wherein the neurodegenerative disorder is Alzheimer's disease.

42. The method of claim 39 wherein CNS levels of neutral endopeptidase-24.11 are increased.

43. The method of claim 39 wherein neuronal or glial levels of neutral endopeptidase-24.11 are increased.

44. The method of claim 39 wherein the fragment comprises an amino acid sequence of an extracellular and a membrane-spanning region of the amyloid precursor protein.

45. The method of claim 39 wherein the fragment comprises a peptide of 38 to 43 amino acid length obtainable by proteolytic cleavage of the amyloid precursor protein using β- and/or γ-secretase.

46. The method of claim 39 wherein the fragment comprises the Aβ₁₋₄₂ peptide.

47. The method of claim 39 wherein the fragment comprises the Aβ₁₋₄₀ peptide.

48. The method of claim 39 wherein the medicament contains the fragment, derivative, or functional mimetic in the form of a monomer or multimer.

49. The method of claim 39 wherein the medicament contains the fragment, derivative, or functional mimetic in aggregated form.

50. The method of claim 39 wherein the medicament contains the fragment, derivative, or functional mimetic in aggregated form as fibrils.

51. The method of claim 39 wherein the fragment, derivative, or functional mimetic is synthetic.


SEQUENCE LISTING (2 sequences)

SEQ ID NO: 1 - DNA, 29 bp, Artificial
  primer_bind
gtggataacccctcccccagcctagacca

SEQ ID NO: 2 - DNA, 25 bp, Artificial
  primer_bind
ctgaccactcgaccaggttctgggt